(19) United States
(12) Patent Application Publication
(10) Pub. No.: US 2005/0053911 A1
(43) Pub. Date: Mar. 10, 2005

(54) Room temperature stable competent cells

(76) Inventors: Alan L. Greener, San Diego, CA (US); James F. Jolly, Wheaton, IL (US)

Correspondence Address: PALMER & DODGE, LLP;KATHLEEN M. WILLIAMS / STR, 111 HUNTINGTON AVENUE, BOSTON, MA, 02199, US

(21) Appl. No.: 10/943,258

(22) Filed: Sep. 15, 2004

Related U.S. Application Data
(63) Continuation of application No. 09/894,806, filed on Jun. 28, 2001, now abandoned.
(60) Provisional application No. 60/255,726, filed on Dec. 15, 2000.

(51) Int. Cl. A01N 1/02; C12N 1/20; C12N 15/74

(52) U.S. Cl. 435/2; 435/471; 435/252.3

(57) ABSTRACT

The invention provides storage-stable competent cells which retain good viability over long periods of time at room temperature (e.g., least one month). In one embodiment, a composition is provided comprising a mixture of competent cells and a glass-forming matrix material. Methods for generating room temperature stable competent cells are also provided.

### RELATED APPLICATIONS

This continuation application claims priority to U.S. application Ser. No. 09/894,806, filed Jun. 28, 2001, which claims priority under 35 U.S.C. § 119(e) to U.S. Provisional Application No. 60/255,726, filed Dec. 15, 2000, the entirety of which are incorporated herein by reference.

### FIELD OF THE INVENTION

The invention relates to competent cells which are stable at room temperature and to methods of generating such cells.

### BACKGROUND OF THE INVENTION

Cells which are primed for the uptake of nucleic acids are referred to as competent cells. These are cells which have been treated to make their cell membranes more permeable in order to facilitate the entry of exogenous nucleic acids. Competent cells serve as vehicles to store and amplify cloned sequences.

Typical methods of generating competent cells comprise growing cells to log phase or early stationary phase and exposing the cells to CaCl₂ at 0° C. (see, e.g., Sambrook, et al., In Molecular Cloning: a Laboratory Manual, 2nd Edition, eds. Sambrook, et al., Cold Spring Harbor Laboratory Press, (1989)). Cells can be contacted immediately with exogenous DNA or frozen in glycerol or DMSO for subsequent use. Upon thawing to 4° C. and contacting with plasmid DNA, frozen competent cells typically have transformation efficiencies of 1×10⁵-1×10⁹ transformants/μg of plasmid DNA.

Electroporation has also been used to transform cells (see, e.g., Dower et al., Nucleic Acids Research, 16: 6127-6145 (1988); Taketo, Biochimica et Biophysica Acta, 949: 318-324 (1988); Chassy and Flickinger, FEMS Microbiology Letters, 44: 173-177 (1987); and Harlander, Streptococcal Genetics, eds. Ferretti and Curtiss, American Society of Microbiology, Washington, D.C., pp. 229-233 (1987)). Electroporation methods rely on creating temporary holes in cell membranes by exposing cells to a high voltage electric impulse to facilitate the uptake of exogenous nucleic acids (see, e.g., Andreason and Evans, Biotechniques, 6: 650-660 (1988)). Cells exposed to an electroporation buffer (e.g., 10-15% glycerol) are generally stored by freezing to provide a supply of electrocompetent cells (see, e.g., U.S. Pat. No. 6,004,804).

Temperatures of −80° C. and below have been used to preserve viability of competent cells (see, e.g., U.S. Pat. No. 4,981,797) since storage at higher temperatures is associated with rapid loss of viability and transformation efficiency within a period of days (see, e.g., Dagent, et al., Gene 6: 23-28 (1979) and Pope, et al., Nucl. Acids Res. 24(3): 536-537 (1996)). However, storage at −80° C. is problematic because of the high cost of equipment necessary to maintain this temperature. It is also difficult to ship competent cells and maintain their viability; generally, competent cells are shipped overnight on dry ice or in the presence of frozen packaging materials, under suboptimal conditions.

Attempts to store competent cells at higher temperatures have been described. U.S. Pat. No. 5,891,692, describes a method of storing competent bacterial cells at −20° C. to 4° C. without appreciably losing transformation efficiency or viability. The method relies on altering the fatty acid content of the bacteria and requires transforming bacterial cells with exogenous E. coli fabB genes.

Jessee, et al., WO 98/35018 disclose a method of lyophilizing competent cells to generate cells which are stable at −20° C. for up to a year. In this method, cells which have been previously frozen from −20° C. to −80° C. are lyophilized in the presence of a cryoprotectant. During lyophilization, the cells are exposed to a series of temperature steps from −45° C. to 10° C. at a rate of about 0.1° C. to 1.0° C./hour. Jessee, et al. report that the cells are stable at a range of temperatures, including room temperature. The competent cells are reported to retain transformation efficiencies of 1×10⁵ to 1×10⁹ transformants/μg of DNA.

### SUMMARY OF THE INVENTION

The invention provides a simple method of generating room temperature competent cells by drying the cells in the presence of a glass-forming matrix. The method comprises growing cells in cell growth media, rendering the cells competent, and drying the cells in the presence of the matrix-forming material under vacuum at a temperature above freezing, preferably from 4° C. to 60° C., and more preferably at 20° C.-40° C.

In one embodiment according to the invention, the glass-forming matrix material comprises a carbohydrate or derivative thereof. Preferably, the glass-forming matrix material is water soluble. In one aspect, the concentration of the carbohydrate or derivative thereof is at least 20% (weight/volume). In another aspect, the glass-forming matrix comprises a saccharide, preferably a non-reducing saccharide. The saccharide can comprise a disaccharide, a trisaccharide, tetrasaccharide, oligosaccharide, polysaccharide, sugar alcohol, sugar ether, sugar acid, and can include derivatives thereof, and combinations thereof. Combinations thereof are ratios of two or more saccharides of, e.g., 1:1, 1:2, 1:5, 1:10, 10:1, 5:1, 2:1 for any two saccharides in the given combination. In a further embodiment of the invention, the sugar is selected from the group consisting of trehalose, sucrose, maltitol, melezitose, raffinose, sorbose, lactitol, dextrose, derivatives thereof, and and combinations thereof. In one aspect, the saccharide is a polysaccharide selected from the group consisting of amylose, Ficoll™, dextrin, starch, dextran, and polydextrose. In some embodiments, the glass forming matrix material comprises a saccharide (e.g., such as trehalose) and a sugar alcohol (e.g., such as sorbitol).

In another aspect, the glass-forming matrix material comprises a polyol (e.g., such as a sugar polyol, propylene glycol, polyethylene glycol, derivatives thereof, and combinations thereof).

In still another aspect, the glass-forming matrix material comprises a polymer selected from the group consisting of polyvinylpyrolidone, polyacrylamide, polyethyleneime, and albumen.

Cells can be rendered competent according to any means known in the art. Preferably, the cells are bacterial cells. More preferably, the cells are gram negative bacterial cells. In one embodiment, cells are rendered competent by exposure to a chemical agent, such as CaCl₂, while in another embodiment, the cells are rendered competent for electroporation by exposure to an electroporation buffer.

In contrast to prior art methods, the method according to the invention does not require exposing cells to a series of temperature steps during the drying process. That is, the cells are exposed to only one temperature during drying. In one embodiment, cells according to the invention are dried for at least two hours at a single uniform temperature (±0.5° C.) and are not subjected to another uniform temperature for a period of time of >5 minutes, and preferably, greater than or equal to at least 2, at least 6, or at least 8 hours. In another embodiment, cells are dried at temperatures above freezing.

In one embodiment, the storage-stable competent cells can be stored at temperatures above −80° C. and maintain transformation efficiencies of at least 10⁵ transformants/μg DNA for at least one month. In another embodiment, the cells can be stored at temperatures of −20° C. or above and maintain transformation efficiencies of at least 10⁵ transformants/μg DNA for at least one month. In still another embodiment, the cells can be stored at temperatures of 0° C., at 4° C., at 15° C., at 20° C., or at room temperature, or above, and maintain transformation efficiencies of at least 10⁵ transformants/μg DNA for at least one month, and preferably, at least about 10⁶ transformants/μg DNA for at least one month.

In one embodiment, competent cells are dried under vacuum (preferably, under pressures of from 1000-3000 mtorr). In a further embodiment, cells are dried under vacuum for 2-24 hours at room temperature (e.g., from 15-30° C.). Still more preferably, cells are dried at 30° C. for 6-48 hours, and preferably for at least 8 hours. In a further embodiment according to the invention, storage-stable competent cells are provided which maintain a transformation efficiency of at least 1×10⁵ transformants/μg DNA for greater than a month at room temperature.

The invention further provides a method of transforming cells with an exogenous nucleic acid comprising: obtaining room temperature storage stable competent cells generated as above, rehydrating the cells (preferably, in transformation buffer or electroporation buffer), and contacting the cells with the nucleic acid. In one embodiment, the method further comprises the step of exposing the cells to at least one electrical pulse.

In a further embodiment, the invention provides a method of producing recombinant polypeptides (e.g., polypeptides expressed by exogenous nucleic acids). In this embodiment, competent cells which have been transformed with a nucleic acid encoding a polypeptide of interest are grown in a cell growth media under conditions in which the nucleic acid will express the polypeptide. The polypeptide is then isolated from the cultured cells and purified.

The invention further provides compositions comprising storage-stable competent cells. In one embodiment the composition comprises a glass-forming matrix material and competent cells, wherein the glass transition temperature (Tg) of the matrix-cell mixture is greater than 15° C., greater than room temperature, greater than 20° C., greater than 30° C., greater than 40° C., greater than 45° C., greater than 50° C., or greater than 60° C. In one embodiment, the transformation efficiency of the cells comprises at least 10⁵ transformants/μg DNA or at least 10⁶-10¹² transformants/μg DNA. In another embodiment, the glass-forming matrix material comprises at least one carbohydrate or derivative thereof. The glass-forming matrix material can be any of the polymers, polyols and/or carbohydrates (e.g., saccharides, saccharide derivatives, and combinations) described above.

In one embodiment, at least 5%, at least 10%, or at least 15% of the storage-stable competent cells are viable upon rehydration. In another embodiment, at least 20% of the cells are viable upon rehydration. In a further embodiment, at least 30% of said cells are viable upon rehydration.

The invention additionally provides kits comprising room temperature stable competent cells which can be shipped to a user without packaging in dry ice or with frozen packaging materials, eliminating costly overnight shipping expenses. In one embodiment, a kit according to the invention comprises a composition comprising a mixture of glass-forming matrix material and cells, wherein the Tg of the mixture is greater than 15° C., greater than room temperature, greater than 20° C., greater than 30° C., greater than 40° C., greater than 45° C., greater than 50° C., or greater than 60° C. In a further embodiment of the invention, the kit comprises a sample of nucleic acids (e.g, such as lyophilized nucleic acids), and optionally, instructions on how to rehydrate the cells and use them in transformation procedures. In a further embodiment, room temperature stable competent cells are packaged in a sealed pouch and optionally provided along with a desiccant, with instructions for reconstituting the cells for transformation. In a further embodiment of the invention, cells are provided along with a sample of supercoiled plasmid DNA, for example, to serve as a control to monitor the transformation efficiency of the competent cells.

### DESCRIPTION

Room temperature stable competent cells provide vehicles for cloning and stably propagating nucleic acids of interest and for producing desired polypeptides. The competent cells according to the invention can be stored long-term (e.g., greater than a month) without the need for a −80° C. storage facility and can be shipped without the use of ice or other frozen packaging materials.

Definitions

In order to more clearly and concisely describe and point out the subject matter of the claimed invention, the following definitions are provided for specific terms which are used in the following written description and the appended claims.

As used herein, “a vector” is a DNA molecule which comprises an origin of replication and is capable of replicating extrachromasomally.

As used herein, the term “polyol” is intended to include any aliphatic or aromatic compound containing at least 2 free hydroxyl groups.

As used herein, a “saccharide” refers to one or more of a disaccharide, trisaccharide, tetrasaccharide, oligosaccharide, polysaccharide and polymers of such saccharides. The term “oligosaccharide” refers to saccharides of from about 5 to about 10 sugar units having molecular weights, when unsubstituted, from about 650 to about 1300. The term “polysaccharide” refers to saccharides comprising greater than about 10 sugar units per molecule.

As used herein, a “derivative” refers to a compound with one or more substituents which still retains the function of the original compound or has improved function. For example, a disaccharide, oligosaccharide, or polysaccharide “derivative” refers to a disaccharide, oligosaccharide, or polysaccharide, respectively, comprising one or more atoms substituted by one or more other atoms, so long as the disaccharide, oligosaccharide, or polysaccharide has one or more, and preferably all, of the properties of being nonreducing or slowly reducing, non-crystallizing upon drying, forming a hydrate when water is absorbed, and comprising a Tg in the range of 10° C. to 80° C. and preferably, in the range of 30° C. to 60° C.

As used herein, “stably stored” refers to cells which are able to withstand storage for extended periods of time (e.g., at least one month, or two, three, four, six, or twelve months or more) with a less than 95%, 90%, 80%, 70%, 60%, 50%, 40%, 30%, 20%, 15%, 10%, 5%, or 1% decrease in viability and which retain a transformation efficiency of at least 1×15 transformants/μg DNA.

As used herein, the term “room temperature” refers to temperatures greater than 4° C., preferably from 15°-40° C., 15° C. to 30° C., and 15° C. to 24° C., and 16° C. to 21° C. Such temperatures will include, 14° C., 15° C., 16° C., 17° C., 18° C., 19° C., 20° C., and 21° C.

As used herein, the term “competent cell” refers to a cell which has the ability to take up and replicate an exogenous nucleic acid, and preferably to produce viable clonal progeny comprising the exogenous nucleic acid.

As defined herein, the term “exogenous DNA” refers to any of: plasmids, cosmids, DNA libraries, cDNA libraries, expression vectors, eukaryotic DNA, phage DNA, phagemid DNA, microbial DNA, single-stranded DNA, double-stranded DNA, supercoiled DNA, circular DNA, linear DNA, and the like.

As defined herein, a “a selectable marker gene” is a gene encoding a marker that can be used to identify the presence of an exogenous DNA in a transformed cell (a cell, or progeny of a cell, which has been contacted with exogenous DNA and which has taken up the DNA). Selectable marker genes include, but are not limited to, drug resistance genes (e.g., antibiotic resistance genes), genes encoding detectable polypeptides (e.g., Green Fluorescent Protein), and genes encoding enzymes (e.g., which can be detected through the catalysis of their substrates), such as β-galactosidase, as well as unique sequences (e.g., producing signature restriction fragments) not found in the genome of the host cell being transformed.

As used herein, a “derivative” of a bacterial strain is a bacterium which comprises one or more mutations compared to a progenitor bacterial strain (e.g., the strain from which the derivative is “derived”) or one or more exogenous sequences compared to a progenitor strain. Mutations can be naturally occurring or induced through exposure to one or more mutagens and/or through the introduction exogenous DNA which recombines with the bacterial genome.

As used herein, the term, “drying at a uniform temperature” refers to exposure of a cells to a constant temperature during the drying process; e.g., without stepwise changes in temperature.

As used herein, the term, “electrocompetent” refers to cells are rendered competent for electroporation by exposure to an electroporation buffer (e.g., such as a solution comprising at least 10% glycerol).

Cells

A variety of cells, prokaryotic and eukaryotic (e.g., cells such as fungi, including yeast), can be rendered competent for transformation. In a preferred aspect, the cells are bacterial cells and include, but are not limited to, gram negative and gram positive bacterial cells, such as Eschericia sp. (e.g., E. coli), Klebsiella sp., Salmonella sp., Bacillus sp., Streptomyces sp., Streptaococcus sp., Shigella sp., Staphylococcus sp., and Pseudomonas sp. In a preferred embodiment of the invention, E. coli strains are rendered competent for transformation by exogenous nucleic acids. Suitable E. coli strains include, but are not limited to, BB4, C600, DH5, DH5a, DH5a-E, DH5aMCR, DH5a5′IQ, DH5a5′, DH10, DH10B, DH10b/p3, DH10BAC, HB101, RR1, JV30, DH11S, DM1, LE392, SCS1, SCS110, Stab2, DH12S, MC1061, NM514, NM522, NM554, P2392, SURE®, SURE 2, STBL2™ Competent Cells or ELECTROMAX™ STBL4™ cells, XL1-Blue, XL1-Blue MRF, XL1-BlueMR, XL2-Blue, JM101, JM109, JM110/SCS110, NM522, TOPP strains, ABLE®, XLI-Red, BL21, TK B1, XL10-Gold® Cells, Restriction-Minus Competent Cells™, TK Cells, ABLE® strain, X1mutS strains, SCS10, AG1, ElectroTen-Blue™ strains, TG1, SOLR™, XLOLR strain, Y1088, Y1089r, Y1090r-strains, WM100, and derivatives thereof. Information relating to the genotypes of these strains are known in the art and can be found, for example, at www.strategene.com.

Methods of Making Competent Cells

Cells are first grown in a medium which supports cell proliferation. Cell growth medium encompassed within the scope of the invention, includes, but is not limited to: Luria Broth; Psi broth (e.g., 5 grams bacto yeast extract, 20 grams Bacto tryptone, 5 grams of magnesium sulfate, per liter); SOB medium (e.g., 0.5% yeast extract, 2% tryptone, 10 mM NaCl, 2.5 mM KCl, 10 mM MgCl₂, 10 mM MgSO₄); SOC medium (e.g., 2% tryptone, 5% yeast extract, 2.5 mM KCl, 10 mM NaCl, 10 mM MgCl₂, 20 mM glucose); Terrific Broth (“TB”) (e.g., 12 grams of tryptone, 24 grams of yeast extract, 4 ml of glycerol 2.3 grams of KH₂PO₄, 12.5 grams of K₂HPO₄, per liter); TY medium (8 grams of tryptone, 5 grams of NaCl, 5 grams of yeast extract, per liter, adjusted to pH 7.2-7.4 with NaOH), and other media used to support the growth of cells, such as bacteria. In one embodiment, the cell growth medium used is supplemented to comprise additional growth-promoting agents (e.g., vitamins, sugars, ions, and the like). It should be obvious to those of skill in the art that a variety of media can be used, and that such media are encompassed within the scope of the invention.

Incubation temperatures for growing cells can vary from 10° C. to 42° C., but preferably ranges from 20° C. to 40° C. In one embodiment according to the invention, cells are grown with shaking to promote aeration, (e.g., at 100 to 500 revolutions per minute (rpms)). In a preferred embodiment, cells are grown to early to mid log phase, or to early stationary phase, as detected by visual inspection (e.g., for optimal turbidity) or by sampling aliquots of media and determining the optical density (OD) of the media (e.g., at 550 nm, using a spectrophotometer) to select cells having an OD⁵⁵⁰ between 0.1 to 2.0, and preferably, between 0.5-2.0. Media can be reinoculated with cells which have reached mid to late stationary phase to reinitiate log phase growth.

In one embodiment, cells at a desired stage of growth are collected (e.g., by centrifugation, filtering, allowing cells to settle, by size exclusion chromatography, and the like) and resuspended, to be washed at least one time, in a suitable transformation buffer. Suitable transformation buffers include, but are not limited to, 50 mM CaCl₂, 10 mM Tris/HCL (Sambrook, et al., Molecular Cloning: a Laboratory Manual, 2nd Edition, eds. Sambrook, et al., Cold Spring Harbor Laboratory Press, (1989)); TB buffer (e.g., 10 mM PIPES, 15 mM CaCl₂, 250 mM KCl) (Inoue, et al, Gene 96: 23-28 (1990)); 2×TSS (LB broth with 10% PEG (MW3350-8000), 5% DMSO, and 20-50 mM Mg²⁺ (MgSO₄ or MgCl₂) at a final pH of 6.5) (Chung, et al., PNAS 86: 2172-2175, (1989)); FSB buffer (e.g., 10 mM-potassium acetate, 100 mM-KCl, 44 mM-MnCl₂, 10 mM-CaCl₂, 3 mM-HACoCl₃, 10% redistilled glycerol) (Hanahan, D., In: DNA Cloning (D. M. Glover, ed) IRL Press, Washington, D.C., pp. 109-135); and CCMB80 buffer (10 mM potassium acetate pH 7.0, 80 mM CaCl₂, 20 mM MnCl₂, 10 mM MgCl₂, 10% glycerol, adjusted to pH 6.4 with 0.1N HCl) (Hanahan, et al., Methods in Enzymology 204: 63-113 (1991)). (The entirety of these references are incorporated herein by reference.) Preferably, the cells are resuspended in transformation buffer which has been pre-cooled to 4° C. (e.g., by chilling on ice) and in one embodiment, cells are incubated in transformation buffer for at least 2-60 minutes.

Cells can also be made competent by exposure to electrical pulses which create temporary holes in the cells' plasma membranes (Potter, Anal. Biochem. 174: 361-73 (1988); U.S. Pat. No. 6,096,549, the entireties of which are incorporated herein by reference). Therefore, in one embodiment, cells are grown in culture media to mid or late log phase or to early stationary phase and collected (e.g., by centrifugation). Cells are then resuspended and washed at least one time in an electroporation buffer (e.g., 10%-15% glycerol, 90% distilled water, v/v), and placed in a chamber of an electroporation device (e.g., Cell-Porator™, from Life-Technologies; BIO-RAD Gene Pulser®), avoiding air bubbles during the placement process. Cells are exposed to an electrical pulse which varies depending upon the cell type and the size of the container in which the cells are placed. In one embodiment, E. coli cells are rendered permeable by exposure to 1.5 to 2.5 kV (25 μF, 200 ohms) (see, e.g., Dower, et al., Nucleic Acids Res. 16: 6127-6145 (1988), the entirety of which is incorporated herein by reference).

Methods of making competent cells can be selected to suit a user's needs. For example, when transforming cells with supercoiled plasmid DNA, generally any method known in the art will provide an acceptable number of transformants (e.g., 1 per agar plate). However, for clones comprising unstable or less stable sequences (e.g., LTR sequences and inverted repeats), it may be desirable to alter growth conditions to enhance the stability of the cells, i.e., such as by growing cells at lower temperatures (25° C. to 30° C.) in rich medium (e.g., TB broth) and by terminating growth before the cells reach late stationary growth phase. Alternatively, or additionally, cells whose genotypes minimize rearrangements of unstable sequences can be used (e.g., such as STBL strains). Where limiting amounts of cloned sequences are to be introduced into a cell, transformation buffers can be additionally supplemented by agents for enhancing transformation efficiency, including, but not limited to, hexamine cobalt chloride, sodium succinate, RbCl, and the like (see, as discussed in U.S. Pat. No. 4,981,797, the entirety of which is incorporated by reference herein).

Additional methods of generating competent cells are described in: Kushner, In: Genetic Engineering: Proceedings of the International Symposium on Genetic Engineering, Elsevier, Amsterdam, pp. 17-23 (1978); Norgard, et al., Gene 3: 279-292 (1978); Jessee, et al., U.S. Pat. No. 4,981,797; and on the world wide web at protocol-online.net/molbio/DNA/transformation.html, the entireties of which are incorporated by reference herein.

Generating Room Temperature Stable Competent Cells

In one embodiment, competent cells prepared by any of the methods described above, or by any methods known in the art, are contacted with a solution comprising a water soluble glass-forming matrix material. In one embodiment, the glass-forming matrix material is hydrophilic and comprises a glass transition temperature (“Tg”) from 10° C. to 80° C. upon drying, and preferably comprises a Tg of at least 40° C., and still more preferably, comprises a Tg of at least 45° C., at least 50° C., or at least 60° C.

Suitable glass-forming matrix materials include carbohydrates, such as non-reducing sugars, which minimize oxidative damage to the cells. In one embodiment, the matrix material is a saccharide selected from the group consisting of trehalose, sucrose, melzitose, raffinose, maltitol, sorbose, lactitol, dextrose, sugar alcohols (e.g., sorbitol, galactitol, mannitol, xylitol, erythritol, threitol, sorbitol glycerol, polyglycerols, such as diglycerol and triglycerol, and the like), sugar ethers (e.g., sorbitan and polyvinyl alcohols), sugar acids (e.g., L-gluconate), and derivatives and combinations thereof. Polysaccharides such as amylose, Ficoll™ (see, U.S. Pat. No. 3,300,474), dextrin, starch, dextran, and polydextrose also can be used.

In a preferred embodiment, the competent cells are contacted with a 20% carbohydrate solution, such as 20% trehalose, 20% sucrose, 20% melzitose, or 20% raffinose. In one embodiment, the cells are exposed to a solution which comprises 10% of two different carbohydrate solutions (e.g., 10% trehalose and 10% melzitose; 10% raffinose and 10% trehalose; 10% raffinose and 10% melzitose; 10% trehalose and 10% sucrose; 10% raffinose and 10% sucrose; or 10% melzitose and 10% sucrose). In one aspect, the matrix material is supplemented with a sugar alcohol, such as sorbitol, preferably at a concentration of 2.5% w/v.

In a preferred embodiment, a saccharide is used which does not crystallize upon drying and which comprises a Tg in the range of 10° C. to 80° C., preferably in the range of 30-80° C., and more preferably in the range of 60-80° C. In one embodiment, the glass forming matrix material is a non-reducing carbohydrate selected from the group consisting of disaccharides, trisaccharides, tetrasaccharides, oligosaccharides, polysaccharides, sugar alcohols, sugar ethers, sugar acids, and derivatives and combinations thereof.

Preferred saccharides include, but are not limited to, trehalose, raffinose, melezitose, sucrose, maltitol, derivatives thereof, and combinations thereof. In one embodiment, a glass-forming saccharide is selected which hydrolyzes into a reducing sugar at a slow rate (e.g., such as trehalose). In another embodiment, a saccharide is selected which forms a hydrate when water is absorbed, thereby maintaining a high Tg (>15° C., preferably greater than 40° C., more preferably, greater than 50° C., and still more preferably, greater than 60° C.) upon drying.

Other glass-forming matrix materials are known and are encompassed within the scope of the invention. These include, but are not limited to, polyols. In addition to sugar polyols, polyols such as propylene glycol and polyethylene glycol, also can be used, as can polymers such as polyvinylpyrolidone, polyacrylamide, polyethyleneimine, albumin, and the like.

Competent cells which have been previously frozen can be used, as well as competent cells which are freshly made (e.g., less than 2 hours old) and have been stored at −20° C. to 4° C. However, preferably, the cells are not frozen immediately prior to drying (i.e., at least 1 minute prior to drying). In one embodiment, the competent cells are collected by centrifugation (e.g., to substantially remove transformation buffer), and resuspended in a solution comprising the glass-forming matrix material. The cells are subsequently dried, for example, by exposure to a vacuum under pressure. Volumes of cells used in the drying process can be modified to suit a user's needs, and can range, for example, from 100 μl (e.g., for small-scale experimental purposes) to 10 liters, for large scale (e.g., commercial) preparations.

Drying can be performed using standard drying apparatuses known in the art as lyophilizes, sublimates, Speedvacs, and the like. Freeze-drying apparatuses can be modified for use in the process (e.g., by not drying in the presence of dry ice, or by setting a temperature control to a temperature above freezing, i.e., such as room temperature or above). The cells themselves are never freeze-dried. In one aspect of the invention, 4 liters of cells are aliquoted into vials at volumes of 150 μl per vial; using a sublimator capable of accommodating 4800 vials.

As water is removed from the competent cells during the drying process, the glass-forming matrix material forms an increasingly glassy amorphous matrix which surrounds the cells. This glassy matrix is stable at temperatures below the Tg; however, at temperatures above the Tg, the glassy matrix loses its structure and becomes more fluid, assuming a syrup-like consistency. Thus, any cells which are stored at temperatures above the Tg of the glass matrix-cell mixture will cease to be immobilized as the glass matrix becomes fluid and will be susceptible to degradation. Since the temperature stability of the glass matrix reflects the storage stable temperature of the cells, in one embodiment, drying conditions are selected which generate a glassy matrix-cell mixture having a Tg which is higher than or equal to the desired storage temperature of the cells, e.g., at least room temperature, preferably greater than 40° C., still more preferably greater than 45° C., greater than 50° C., and most preferably, greater than 60° C.

In one embodiment, optimal temperature and drying times are identified by drying cells, measuring the Tg of the glassy matrix-cell mixture as the drying process proceeds, and subsequently standardizing the drying procedure to achieve a desired Tg (i.e., a Tg at least as high as a desired storage and/or shipping temperature).

The Tg of the glass matrix-cell mixture can be measured using means standard in the art, such as by differential scanning calorimetry, dynamic thermal analysis (DTA), dynamic mechanical thermal analysis (DMTA), dynamic mechanical analysis (DMA), low field NMR., and the like. In one embodiment, the Tg of the matrix is determined at different time points during the drying process, to determine whether additional drying is required. It should be obvious to those of skill in the art that while different methods of measuring Tg's may yield slightly different results, whether the cell-glass matrix mixture has reached a desired Tg also can be verified empirically, e.g., by determining whether the composition remains in a glassy form at a selected temperature, such as room temperature or higher. Thus, a cell having a Tg of a given temperature would remain in its glassy form (i.e., not liquefy) at temperatures of the given temperature and below.

In one aspect of the invention, the Tg of the matrix-cell mixture is increased by adding a glass-enhancing agent, e.g., such as a zwitterion comprising polar or apolar radicals, such as amino carboxylic acids, and their salts (see, EP-913178, the entirety of which is incorporated herein by reference), zinc ions or other metal ions (see, e.g., U.S. Pat. No. 4,806,343, the entirety of which is incorporated herein by reference), and borate. In another aspect, the glass-matrix material comprises a saccharide which is already hydrated (see, e.g., U.S. Pat. No. 6,071,428, the entirety of which is incorporated by reference herein) prior to being contacted with the competent cells to increase the Tg of the glass-matrix: cell mixture.

In one embodiment according to the invention, drying conditions are selected which provide a Tg of greater than or equal to 20° C., greater than or equal to 25° C., greater than or equal to 30° C., greater than or equal to 35° C., preferably greater than or equal to 45° C., and more preferably, greater than or equal to 50° C. In a more preferred embodiment, conditions are selected which result in a Tg of greater than or equal to 60° C.

In one embodiment, drying is performed using a stepwise temperature increase from 4° C. to 40° C. (i.e., above freezing) over a period of 48 hours. However, room temperature stable competent cells can be generated without temperature steps, and viability is actually enhanced 30% upon drying at a uniform temperature, as measured by plating cells which have been dried using temperature steps, counting the number of colonies formed, and comparing these numbers to the numbers of colonies formed from plated cells which have not been exposed to temperature steps. In one embodiment, cells are dried at room temperature. In a preferred embodiment, cells are dried at a temperature within the range of from 15° C.-40° C. In a more preferred embodiment, cells are dried at 30° C.

Drying times can be varied to achieve an optimal Tg. In one embodiment, the drying time ranges from 2-48 hours. In a preferred embodiment, the drying time ranges from 6-24 hours. More preferably, the drying time is at least 8 hours. In one embodiment of the invention, cells are dried at 30° C. from 18-24 hours, such that a drying run can be easily set up overnight while cells are prepared (i.e., rendered competent) during the day.

In one embodiment, competent cells are dried under vacuum, to maximize the amount of matrix-cell mixture formed in the minimum amount of time, thereby maximizing cell viability. In one embodiment, it is preferred that the matrix-forming material-cell mixture be dried at atmospheric pressure. Pressure is optimized to provide the highest Tg and cell viability while providing a product that dries in an intact form (e.g., without forming bubbles). In a preferred embodiment of the invention, the glass-matrix-cell mixture is dried at 1000-3000 mtorr, and preferably at 2000 or 3000 mtorr. Pressure can be optimized in consideration of the surface area of cells being dried, e.g., to take into account the type of container the cells are being dried in. After drying is completed and a satisfactory Tg is obtained, dried cells are stored in sterile containers at room temperature until use. Where cells are dried in bulk, dried cells can be milled into powder and dispensed into individual containers suitable for use. In one embodiment according to the invention, cells are packaged in a form suitable for shipping, for example, by storing the cells in sealed pouches in the presence of desiccant.

Post-Drying Processing Steps: Transforming Room Temperature Competent Cells

Dried cells can be rehydrated for use in subsequent transformation procedures. In one embodiment, the dried competent cells are resuspended in an appropriate amount of water which does not lyse the cells; i.e., generally, at least a volume of water equal to the volume of stored competent cells. Cells may be further diluted in buffer (e.g., transformation buffer) or cell growth media. In one embodiment, cells are rehydrated, collected (e.g., by centrifugation), and washed at least one time in a transformation medium or cell growth medium, to remove or substantially dilute, residual glass matrix forming material (e.g., to 5% w/v or less). Still more preferably, cells are immediately resuspended in an equal volume of chilled transformation buffer (e.g., such as FSB) or electroporation buffer, such that both immediately prior to drying and after drying, the cells are in contact with transformation buffer or electroporation buffer.

In one embodiment, rehydrated room temperature stable competent cells according to the invention are used in transformation procedures by contacting the cells with nucleic acids, preferably comprising a selectable marker gene (e.g., a gene encoding resistance to an antibiotic or expressing a detectable polypeptide, or enzyme which can catalyze a detectable reaction, such as β-galactosidase), and plating the cells on a plate which comprises a selection media (e.g., an antibiotic or substrate for the enzyme).

Nucleic acids encompassed within the scope of the invention, include, but are not limited to, nucleic acid sequences that encode functional or non-functional peptide, polypeptides, proteins and fragments of those sequences, as well as nucleic acids which comprise non-coding sequences (e.g., regulatory sequences, such as promoters or enhancers). The nucleic acids may be natural (e.g., isolated from cells) or synthetic nucleic acids (e.g., obtained by PCR or mutagenesis of isolated nucleic acids, or chemically synthesized). The nucleic acids can be circular, linear, or supercoiled. Although not limited to particular sizes, in some embodiments, the nucleic acids used to transform the cells according to the invention range from 1.0 kb to 300 kb.

In one embodiment, competent cells which have been contacted with nucleic acids are incubated for 2 minutes to 2 hours at 4° C.-30° C. For chemically competent cells, preferably, a volume of cells rehydrated in transformation buffer are transferred to pre-chilled tubes and chilled on ice (e.g., are at 4° C.) for ten minutes, and incubated in the presence of exogenous DNA on ice for an additional 20 minutes. Contacted cells are plated onto agar plates comprising a suitable selection media, either directly, or after dilution in a cell growth medium (which can also be further incubated to promote cell growth). In one embodiment of the invention, cells are heat shocked at 20-42° C. for 30 seconds to 2 minutes, prior to plating. Preferably, cells are heat shocked at 42° C. for 60 seconds, transferred to ice for 2 minute, and diluted in culture medium (e.g., generally, a 10:1 dilution). Cells are preferably incubated with aeration (e.g., shaking) for 1 to 10 hours prior to plating (preferably, 2 hours) and then plated onto a solidified culture medium comprising the appropriate selection medium (e.g., antibiotics or a substrate if the exogenous DNA expresses an enzyme capable of catalyzing a substrate).

Transformation efficiencies of the storage stable cells generated according to the method range from at least 10⁵ to 10⁹ transformants/μg DNA, and preferably, at least from 10⁶ to 10⁹ transformants/μg DNA, while the viability of the cells comprises at least 5%, and preferably, at least 10-15% of the viability of cells prior to drying. In one embodiment, the viability of the cells comprises at least 20%, or at least 30%, of the viability of cells prior to drying.

In a further embodiment of the invention, electrocompetent storage stable cells are rehydrated (e.g., in electroporation buffer) and exposed to one or more electrical pulses (1.5-2.5 kV) in the presence of nucleic acids. As above, transformed cells can be directly plated or plated after dilution in cell culture media. In one embodiment, the cells are gently resuspended in SOC medium (e.g., 2 ml of 20% glucose and 1 ml of 2M Mg per 100 ml of SOB medium) after electroporation.

In one embodiment, transformation efficiencies of electrocompetent storage stable cells range from 10⁵ to 10¹² transformants/μg DNA, while the viability of the cells comprises at least 10% of the viability of cells prior to drying. In another embodiment, the viability of the cells comprises at least 15%, at least 20% or at least 30% of the viability of cells prior to drying. In one embodiment, the transformation efficiency of rehydrated cells which have been exposed to at least one electrical pulse is increased relative to cells which have not been subject to drying and rehydration. In another embodiment, transformation efficiencies of the cells are at least three times as great as the transformation efficiency of cells which have not been subjected to drying and rehydration.

Additional factors can be manipulated to enhance the transformation efficiency of pulsed cells such as the electrical field strength, the pulse decay time, the pulse shape, the temperature at which electroporation is conducted, the type of cell (e.g., SURE™ cells and XL1-Blue MRF′™ are particularly suited for electroporation), the type of suspension buffer, and the concentration and size of the nucleic acid to be transferred. Optimization parameters are discussed, for example, in Andreason and Evans, Analytical Biochemistry 180: 269-275 (1988); Sambrook, et al., In Molecular Cloning: a Laboratory Manual, 2nd Edition, eds. Sambrook, et al. (Cold Spring Harbor Laboratory Press) pp. 1.75 and 16.54-16.55 (1989); Sambrook, et al. 1987; Stratagene Instruction Manual for Epicurian Coli™ Electroporation-Competent Cells 1997; the entireties of which are incorporated by reference herein). In one embodiment, additional sugars are added to enhance the electroporation efficiency of the cells (e.g., 0.1% and 5.0% w/v of non-polar aldoses and aldose alcohols).

Producing Recombinant Polypeptides Using Storage Stable Competent Cells

In a further embodiment, the invention provides a method of producing recombinant polypeptides (e.g., polypeptides expressed by the exogenous nucleic acids which have been used to transform the cells). In this embodiment, competent cells which have been transformed with a nucleic acid encoding a protein of interest are grown in a cell growth media under conditions in which the cell will express the polypeptide (e.g., the polypeptide may be expressed constitutively by the cell or under inducing conditions, such as during exposure to a selected temperature or a chemical agent, such as IPTG). The polypeptide is then isolated from the cultured cells and purified, e.g., by lysing the cells (e.g., with lysozyme, exposure to a detergent, by sonication, or by some other method), fractionating cellular components, and selecting for fractions of these components which have any of: a desired enzymatic activity, immunological activity, physical characteristics (e.g., molecular mass, spectroscopic properties, and the like), and/or other biological activity.

Fractionating can be performed using affinity column chromatography where an antibody is available for a polypeptide/antigen of interest, by size exclusion chromatography to select polypeptides within a certain size range, by ammonium sulfate precipitation, polyethylene glycol precipitation, or by using combinations of these methods. Methods of purifying recombinant polypeptides from bacterial cells are well known in the art (see, e.g., Sambrook, et al., supra, and www.protocol online.net/molbio/Protein/protein_purification.htm#Protein Extraction).

Kits

The invention further provides kits comprising room temperature stable competent cells. In one embodiment according to the invention, a kit is provided which comprises room temperature stable competent cells in a container for shipping which does not comprise ice or any other frozen packing material. In another embodiment of the invention, room temperature stable competent cells are packaged in a sealed pouch and optionally provided along with a desiccant. In another embodiment, the cells can be stored in a closed/sealed moisture barrier, or a rigid/sealed container in the presence of desiccant. A variety of desiccants can be used to reduce the water content of the cells, including, but not limited to, calcium sulfate, silica, certain clays, polyacrylic acid, and derivatives thereof.

In a further embodiment of the invention, cells are provided along with a sample of plasmid DNA (e.g., such as lyophilized and/or supercoiled plasmid DNA) which serves as a control to monitor the transformation efficiency of the competent cells. Additional reagents can also be provided for use in transforming the competent cells, such as a substrate for a marker enzyme which is expressed by a nucleic acid to be transformed (e.g., X-Gal), antibiotics, restriction enzymes to detect signature restriction sites in a cloning vector, and the like.

### EXAMPLES

The invention will now be further illustrated with reference to the following examples. It will be appreciated that what follows is by way of example only and that modifications to detail may be made while still falling within the scope of the invention.

### Example 1

E. coli XL Blue MRF cells were grown in LB media (100 ml) to OD⁵⁵⁰ of 0.7-0.75 and then placed on ice for 15-30 minutes. Cells were transferred to four chilled centrifuge tubes and centrifuged for 5 minutes at 5000 RPM, 4° C. Cell pellets were resuspended in ice cold 20% trehalose (25 ml for each pellet). The cells were then centrifuged and washed with 20% trehalose two additional times. Control cells were frozen at −80° C. after the addition of 10% glycerol. Cells to be dried were placed onto the surface of metal cups in 40 μl aliquots. The cups were placed onto a pre-chilled shelf (4° C.) in a Lyostar (FTS Kinetics) lyophilizer. The cells were dried under 3000 mtorr vacuum at the following temperatures: 600 minutes at 4° C., 600 minutes at 10° C., 600 minutes at 15° C., 600 minutes at 20° C., 600 minutes at 25° C. and 600 minutes at 30° C. At the end of drying the cells were resuspended in 40 μl of water and titrated to determine cell viability compared to control cells frozen at −80° C. The dried cells maintained 1% viability compared to control cells.

### Example 2

Cells were prepared as in example 1, and dried for 2 hours at 30° C., 3000 mtorr in a Lyostar lyophilizer. Dried cells were resuspended in 40 μl of water and titrated for viability compared to control cells frozen at −80° C. that were not dried. Results indicated that the dried cells maintained 10% viability compared to control cells. When the cells were tested for electrocompetency, it was found that the dried cells were 3.5 fold more electrocompetent than control cells when the difference in viability was considered (dried cells gave 35% of control number of transformants).

### Example 3

Competent E. coli XL 10 blue cells were dried according to example 2. Cell viability of dried cells were found to be 10% of control cells and the dried cells were about 80% as chemically competent as control cells on a per cell basis.

### Example 4

Cells were dried at 30° C., 3000 mtorr vacuum and for increasing amounts of time. Dried cells were removed from the lyophilizer at 2 hours, 4 hours, 16 hours and 24 hours. Cell viability was determined and compared to control cells frozen at −80° C. (not dried). Cell viability was as follows: 2 hours, 61%; 4 hours, 53%; 16 hours, 7%; and 24 hours, 3%. Glass transition temperatures (Tg) were determined for the dried cells and found to be: 2 hours, 31.7° C.; 4 hours, 51.5° C., 16 hours, 68.9° C. and 24 hours, 74° C.

### Example 5

E. coli XA49 were dried as described in example 4. Dried cells were removed from the lyophilizer at 2 hours, 4 hours, 8 hours, 15 hours and 22 hours. Cell viability compared to control cells (not dried) stored at −80° C. were as follows: 2 hours, 70%; 4 hours, 90%; 8 hours, 90%, 15 hours, 20% and 22 hours, 15%.

### Example 6

E. coli XL Blue cells were prepared as described in example 1, aliquoted in 2 ml glass vials and dried overnight at 30° C., 3000 mtorr in the lyophilizer. After drying the vials were capped and placed in zip lock aluminum pouches with desiccant and stored either at 4° C. or room temperature. At the indicated time period after drying, vials are removed from the pouches, re-suspended in water and the viability was determined and compared to viability immediately after drying. The results are shown in Table 1, below.

TABLE 1
Viability of Competent Cells After Storage

| Days Storage at Room Temperature | Transformation Efficiency |
|---|---|
| 2 | 2.5 × 10⁶ (100%) |
| 40 | 4.2 × 10⁶ (168%) |
| 60 | 2.5 × 10⁶ (100%) |

Variations, modifications, and other implementations of what is described herein will occur to those of ordinary skill in the art without departing from the spirit and scope of the invention as claimed. Accordingly, the invention is to be defined not by the preceding illustrative description but instead by the spirit and scope of the following claims.

1. A method for generating storage-stable competent cells, which comprises drying competent cells at a temperature greater than freezing so as to generate storage-stable competent cells, wherein said cells are not freeze-dried.

2. The method of claim 1, wherein said cells are bacterial cells.

3. The method of claim 2, wherein said bacterial cells are gram negative cells.

4. The method of claim 1, wherein said cells are made competent by exposure to an electroporation buffer.

5. The method of claim 1, wherein the cells are dried for a single uniform temperature.

6. The method of claim 1 or 5, wherein the cells are dried for at least 8 hours.

7. The method of claim 1, wherein the cells are made competent by exposure to a chemical agent.

8. The method of claim 7, wherein said chemical agent is CaCl₂.

9. The method of claim 1, wherein said competent cells can be stored at temperatures above −80° C. for at least one month and maintain transformation efficiencies of at least 10⁵ transformants/μg DNA.

10. The method of claim 9, wherein said competent cells can be stored at temperatures of −20° C. or above for at least one month and maintain transformation efficiencies of at least 10⁵ transformants/μg DNA.

11. The method of claim 10, wherein said competent cells can be stored at temperatures of 0° C. or above for at least one month and maintain transformation efficiencies of at least 10⁵ transformants/μg DNA.

12. The method of claim 11, wherein said competent cells can be stored at temperatures of 4° C. or above for at least one month and maintain transformation efficiencies of at least 10⁵ transformants/μg DNA.

13. The method of claim 12, wherein said competent cells can be stored at temperatures of 15° C. or above for at least one month and maintain transformation efficiencies of at least 10⁵ transformants/μg DNA.

14. The method of claim 11, wherein said competent cells can be stored at temperatures of 20° C. or above for at least one month and maintain transformation efficiencies of at least 10⁵ transformants/μg DNA.

15. The method of claim 1, wherein said cells are dried at a temperature above 0° C.

16. The method of claim 15, wherein said cells are dried at a temperature above 4° C.

17. The method of claim 16, wherein said cells are dried at a temperature at or above room temperature.

18. The method of claim 17, wherein said cells are dried at 30° C.

19. The method of claim 1, wherein said competent cells are exposed to non-atmosphere pressure during drying.

20. The method of claim 1, wherein said competent cells are dried under vacuum.

21. The method of claim 1, wherein said competent cells are dried in the presence of a glass-forming matrix material.

22. The method of claim 21, wherein said glass-forming matrix material comprises at least one carbohydrate.

23. The method of claim 22, wherein said at least one carbohydrate comprises a saccharide.

24. The method of claim 21, wherein the glass-forming matrix is water-soluble.

25. The method of claim 23, wherein said saccharide is selected from the group consisting of a disaccharide, an oligosaccharide, a polysaccharide, a sugar alcohol, a sugar ether, a sugar acid, derivatives thereof, and combinations thereof.

26. The method of claim 23, wherein said saccharide is a non-reducing sugar.

27. The method of claim 23, wherein said saccharide is selected from the group consisting of trehalose, sucrose, melzitose, raffinose, maltitol, sorbose, lactitol, dextrose, derivatives thereof, and combinations thereof.

28. The method of claim 23, wherein said saccharide is a polysaccharide is selected from the group consisting of amylose, FICOLL™, dextrin, starch, dextran, and polydextrose.

29. The method of claim 21, wherein said glass-forming matrix material comprises a polyol.

30. The method of claim 29, wherein said polyol is selected from the group consisting of a sugar polyol, propylene glycol, polyethylene glycol, derivatives thereof, and combinations thereof.

31. The method according to claim 21, wherein said glass-forming matrix material comprises a polymer selected from the group consisting of polyvinylpyrolidone, polyacrylamide, polyethyleneimine, and albumen.

32. The method of claim 22, wherein the concentration of said carbohydrate is at least 20% (weight/volume).

33. The method according to claim 22, wherein said carbohydrate comprises a saccharide and a sugar alcohol.

34. The method according to claim 33, wherein said saccharide is trehalose.

35. The method according to claim 33 or 34, wherein said sugar alcohol is sorbitol.

36. The method according to claim 33, wherein said saccharide is a hydrated saccharide.

37. The method of claim 36, further comprising the step of storing said competent cells at a temperature at or above −20° C.

38. The method of claim 37, further comprising the step of storing said competent cells at a temperature at or above 0° C.

39. The method of claim 38, further comprising the step of storing said competent cells at a temperature at or above 4° C.

40. The method of claim 39, further comprising the step of storing said competent cells at a temperature at or above 15° C.

41. The method of claim 40, further comprising the step of storing said competent cells at a temperature at or above room temperature.

42. The method of claim 37, wherein said competent cells are stored in a sealed pouch.

43. A method of transforming cells with exogenous nucleic acids comprising, obtaining cells generated according to the method of claim 1, rehydrating the cells, and contacting the cells with said nucleic acids.

44. The method of claim 43, further comprising the step of exposing the cells to at least one electrical pulse.

45. The method of claim 43, wherein said cells are rehydrated in transformation buffer or electroporation buffer.

46. The method of claim 43 or 44, wherein said cells exhibit transformation efficiencies of at least 1×10⁵ transformants/μg DNA.

47. A composition comprising a glass-forming matrix material and competent cells, wherein the glass transition temperature (Tg) of the matrix-cell mixture is greater than 15° C.

48. The composition of claim 47, wherein the glass transition temperature (Tg) of the matrix-cell mixture is greater than room temperature.

49. The composition of claim 48, wherein the glass transition temperature (Tg) of the matrix-cell mixture is greater than 20° C.

50. The composition of claim 49, wherein the glass transition temperature (Tg) of the matrix-cell mixture is greater than 25° C.

51. The composition of claim 50, wherein the glass transition temperature (Tg) of the matrix-cell mixture is greater than 30° C.

52. The composition of claim 51, wherein the glass transition temperature (Tg) of the matrix-cell mixture is greater than 40° C.

53. The composition of claim 52, wherein the glass transition temperature (Tg) of the matrix-cell mixture is greater than 45° C.

54. The composition of claim 53, wherein the glass transition temperature (Tg) of the matrix-cell mixture is greater than 50° C.

55. The composition of claim 54, wherein the glass transition temperature (Tg) of the matrix-cell mixture is greater than 60° C.

56. The composition of claim 47, wherein the transformation efficiency of said cells comprises at least 10⁵ transformants/μg DNA.

57. The composition of claim 47, wherein the transformation efficiency of said cells comprises at least 10⁶ transformants/μg DNA.

58. The composition of claim 47, wherein said glass-forming matrix comprises at least one carbohydrate.

59. The composition of claim 58, wherein said carbohydrate comprises a saccharide.

60. The composition of claim 59, wherein said saccharide is selected from the group consisting of a disaccharide, an oligosaccharide, a polysaccharide, a sugar alcohol, a sugar ether, a sugar acid, derivatives thereof, and combinations thereof.

61. The composition of claim 59, wherein said saccharide comprises a non-reducing sugar.

62. The composition of claim 59, wherein said saccharide is selected from the group consisting of trehalose, sucrose, melzitose, raffinose, maltitol, sorbose, lactitol, dextrose, derivatives thereof, and combinations thereof.

63. The composition of claim 59, wherein said saccharide comprises a polysaccharide selected from the group consisting of amylose, FICOLL™, dextrin, starch, dextran, and polydextrose.

64. The composition of claim 58, wherein said carbohydrate comprises a saccharide and a sugar alcohol.

65. The composition of claim 64, wherein said saccharide comprises trehalose.

66. The composition of claim 64, wherein said sugar alcohol comprises sorbitol.

67. The composition of claim 47, wherein said glass-forming matrix material comprises a polyol.

68. The composition of claim 67, wherein said polyol is selected from the group consisting of a sugar polyol, propylene glycol, polyethylene glycol, derivatives thereof, and combinations thereof.

69. The method according to claim 47, wherein said glass-forming matrix material is a polymer selected from the group consisting of polyvinylpyrolidone, polyacrylamide, polyethyleneimine, and albumen.

70. The composition of claim 47, wherein at least 5% of said cells are viable upon rehydration.

71. The composition of claim 70, wherein at least 10% of said cells are viable upon rehydration.

72. The composition of claim 71, wherein at least 15% of said cells are viable upon rehydration.

73. The composition of claim 72, wherein at least 20% of said cells are viable upon rehydration.

74. The composition of claim 71, wherein at least 30% of said cells are viable upon rehydration.

75. A kit comprising a composition according to claim 47, wherein said matrix-cell mixture is stored in a sealed pouch.

76. The kit of claim 75, wherein the kit further comprises a sample of nucleic acids in a container which is separated from said sealed pouch.

77. The kit according to claim 75, wherein said nucleic acids are lyophilized.

78. A method of producing a recombinant polypeptide comprising:
obtaining cells generated according to the method of claim 1;
rehydrating the cells;
contacting the cells with a nucleic acid encoding said recombinant polypeptide; and
growing said cells in a cell growth media under conditions in which the cells produce said polypeptide.

79. The method of claim 78, in which cells which have taken up said nucleic acid are separated from cells which have not taken up said nucleic acids.

80. The method of claim 78, wherein said recombinant polypeptide is isolated from said cells.
